(19) United States
(12) Patent Application Publication
(10) Pub. No.: US 2026/0125352 A1
(43) Pub. Date: May 7, 2026

(54) PROCESSES FOR CONVERTING FURFURALS

(71) Applicant: Origin Materials Operating, Inc., West Sacramento, CA (US)

(72) Inventors: Petar LACHKOV, West Sacramento, CA (US); Junnan Shangguan, West Sacramento, CA (US); Gabriel Viana Sueth Seufitelli, West Sacramento, CA (US); Siavash Fadaeerayeni, West Sacramento, CA (US); Jeremy Dedard, West Sacramento, CA (US); Gaurav Agrawal, West Sacramento, CA (US); Andrew Palermo, West Sacramento, CA (US); Madhu Anand, West Sacramento, CA (US); Dario Prieto-Centurion, West Sacramento, CA (US); Aditya Rane, West Sacramento, CA (US); Kate Elizabeth Ann Pearson, West Sacramento, CA (US); Fan Lin, West Sacramento, CA (US); Richard Todd Emery, West Sacramento, CA (US)

(21) Appl. No.: 19/421,918

(22) Filed: Dec. 16, 2025

Related U.S. Application Data
(63) Continuation of application No. PCT/US24/35291, filed on Jun. 24, 2024.
(60) Provisional application No. 63/522,993, filed on Jun. 23, 2023.
(60) Provisional application No. 63/522,988, filed on Jun. 23, 2023.
(60) Provisional application No. 63/596,887, filed on Nov. 7, 2023.

(51) Int. Cl. C07D 307/46

(52) U.S. Cl. CPC C07D 307/46

(57) ABSTRACT

Furfurals biomass conversion products may be converted to various useful intermediate compounds such as 5-methylfurfural, 5-methylfurfuryl alcohol, 2,5-dimethylfuran, and p-xylene. Various processes are described to convert biomass conversion product 5-chloromethylfurfural into 5-methylfurfural, 5-methylfurfuryl alcohol, 2,5-dimethylfuran, and/or p-xylene. Various processes are described to convert 5-methylfurfural may be converted to 5-methylfurfuryl alcohol, 2,5-dimethylfuran, and p-xylene.

### CROSS REFERENCE TO RELATED APPLICATIONS

This application is a continuation of International Application No. PCT/US2024/35291, filed Jun. 24, 2024, which claims the benefit of U.S. Provisional Application No. 63/522,988, filed Jun. 23, 2023; U.S. Provisional Application No. 63/522,993, filed Jun. 23, 2023; and U.S. Provisional Application No. 63/596,887, filed Nov. 7, 2023; each of which are herein incorporated by reference in their entirety.

### FIELD

The present disclosure relates to the production of useful intermediate compounds from biomass conversion products, and more specifically to the production of furfurals, and more specifically to the production of 5-methylfurfural, 5-methylfurfuryl alcohol, 2,5-dimethylfuran and p-xylene from 5-chloromethylfurfural and/or 5-methylfurfural.

### BACKGROUND

The shift from fossil resources to decarbonized materials is creating an abundance of potential sources for useful chemical compounds. Depending on the processes involved, converting biomass to useful materials can generate byproducts or waste streams that include useful chemical compounds. The challenge thus far has been developing commercially viable processes to capture these useful chemical compounds, at a sufficient yield and cost.

The compound 5-methylfurfural (“MF”) is a common byproduct from biomass conversion processes, including from 5-chloromethylfurfural (CMF) derived from biomass carbohydrates and produced in high yields directly from biomass. CMF is a precursor to several valuable chemicals, including MF and its alcohol, 5-methylfurfuryl alcohol (“MFA”).

Furan aldehydes and their alcohol analogs have promise as initial platforms for a wide array or useful chemical intermediates and fuels. MF is a promising initial platform for a wide array of useful chemical intermediates and fuels, provided that it can be produced at a high purity via a commercially viable process, and without excessive reactants or undesirable byproducts. One such intermediary One such intermediary is the heterocyclic compound 5-methylfurfuryl alcohol (MFA), a reactive furan intermediate that can be used in fine chemical synthesis. Another intermediary is the heterocyclic compound 2,5-dimethylfuran (“DMF”). DMF has an energy density about 40% greater than ethanol, making DMF an attractive biofuel. The compound is chemically stable and insoluble in water. DMF also has utility as a platform for other valuable chemical intermediaries. One example is the aromatic hydrocarbon p-xylene (“pX”), a well-known chemical feedstock in high demand. However, prior art processes for converting biomass conversion byproducts such as CMF and MF into intermediaries such as MFA, DMF and pX have struggled with sufficient yield and commercial viability.

Previous approaches to convert CMF to more useful intermediaries have failed in terms of high purity via a commercially viable process and avoiding excessive reactants or undesirable byproducts, among other problems. For example, U.S. Pat. No. 4,335,049, granted Jun. 15, 1982 and incorporated by reference in its entirety, describes a hydrodechlorination or reduction reaction of 5-chloromethylfurfural (CMF), to produce 5-methylfurfural (MF) over a Pd catalyst and a basic additive such as a tertiary amine. However, this disclosure does not sufficiently describe a complete process to produce MF from CMF. When CMF is reduced to MF as described in this disclosure, HCl is produced, which reacts with the tertiary amine base to form an amine-HCl salt. The amine-HCl salt is detrimental to the function and lifespan of the Pd catalyst, resulting in exceptionally unfavorable turnover numbers (“TON,” a ratio of the number of moles of a product produced by a mole of catalyst before the catalyst becomes inactivated). For example, this references states that amount of palladium in a batch reaction is 0.001 to 1 mole, and preferably 0.0005 to 0.10 mole, to 1 mole of CMF. This equates to a TON of 1-1000, and preferably 10-200. At such low turnovers, the process becomes commercially untenable. Additionally, tertiary amines react with CMF to form undesirable quaternary ammonium salts (amine-CMF salts) via the Menshutkin reaction. Amine-CMF salts are generally insoluble in hydrocarbons and form solids that plug reactors and cause expensive process disruptions. Additionally, this reference is entirely silent on recovering the amine for reuse.

What is needed, then, are processes to convert biomass conversion byproducts—furfurals such as CMF, MF, and MFA, into intermediaries such as MFA, DMF, and pX, at sufficient yields and purities, and with acceptable turnover numbers, to achieve commercial viability.

It is an object of this disclosure to describe processes for converting CMF to MF, MFA, DMF, and/or pX. It is also an object of this disclosure to describe processes for converting MF and/or, MFA to DMF, and/or pX.

It is an object of this disclosure to describe processes for producing MF and/or MFA in which HCl is neutralized as it forms, thereby protecting the function and increasing the lifespan of the Pd catalyst.

It is also an object of this disclosure to describe processes for producing MF and/or MFA in which the production of undesirable quaternary ammonium salts is minimized or eliminated.

It is also an object of this disclosure to describe processes for producing MF and/or MFA in amine base additives are recovered and available for recycling through the process.

### BRIEF SUMMARY

This disclosure relates to the extraction of valuable chemical intermediaries from furfural byproducts of biomass conversion. In particular, the present approach relates to processes for converting CMF into MF and/or MFA; MF into MFA; and CMF, MF, and/or MFA into DMF and optionally into pX.

In embodiments of the present approach for converting CMF, a CMF stock (e.g., from a biomass conversion process) in an organic solvent (e.g., toluene) is reacted with an amine base additive in the presence of hydrogen to produce an effluent having MF, the amine base additive, and at least one amine-HCl salt. The reaction preferably proceeds over a catalyst, and more preferably a Pd catalyst. In some embodiments, the amine-HCl salts are neutralized to produce an MF and amine base additive composition which can be decanted to produce an MF-rich phase and an amine-rich phase. The amine-HCl salt may be neutralized using a caustic agent. In such embodiments, the amine-rich phase can be distilled to produce a purified amine base additive and an amine-MF distillate, the latter of which may be recycled to the decanting process. Also, the MF-rich phase may be distilled to produce a purified MF and an amine-MF distillate, the latter of which may be recycled to the decanting process. It should be appreciated that the CMF to MF reaction may proceed in one or more batch reactors, and/or one or more continuous reactors. In the present approach, a reactor may be a back-fed reactor.

Some embodiments involve a CMF stock that contains organic soluble materials (“OSMs”). In some embodiments the OSMs remain in a solution with MF after separation of the organic solvent and the amine base additive. The MF and the OSMs may be separated using a heavy solvent to prevent or minimize OSM solidification.

Under the present approach, the amine base additive may be a tertiary amine of the formula R₃N, wherein each R may be the same or different and is a straight chain alkyl or a branched chain alkyl, and preferably wherein the average number of carbon atoms in each R is from 1 carbon atoms to 12 carbon atoms. For example, the amine base additive may be one or more of: tri-n-butyl amine (“TBA”), tri-isobutyl amine (“TiBA”), tris-2-ethylhexyl amine (“TEHA”), trihexylamine (“THEX”), dimethyldocecylamine (“DIMLA”), dibutylaniline (“DBAN”), trimethylamine (“TMA”), N, N-diisopropylethylamine (“DIPEA”), tri-n-amylamine, tri-octylamine, and triethylamine (“TEA”).

The organic solvent may be one or more of a polar organic solvent, methanol, acetone, dimethylformamide, MF, tetrahydrofuran, furfural, a non-polar organic solvent, benzene, alkylbenzene, xylene, and toluene. The 5-chloromethylfurfural stock preferably comprises a CMF concentration of less than 60 wt %, or less than 50 wt %, or less than 40 wt %, or less than 30 wt %, or less than 20 wt %, or less than 15 wt %, or less than 10 wt %, or less than 5%, with these concentrations being ±1 wt %.

The present approach may feature one or more separation operations following the CMF to MF reaction, and in some embodiments after amine-HCl salt neutralization. For example, a first separation may remove organic solvent, leaving MF, the amine base additive, and any OSMs for subsequent separation operations. MF and amine base additives may be separated through decanting to produce amine-rich and MF-rich phases. Those phases, in turn, may be distilled to produce pure bottoms products, and the distillate products may be returned to the decanting operation.

In some embodiments, the reactor effluent is mixed washed with dilute HCl to produce an acid-washed mixture of an organic phase having MF and the organic solvent, and an aqueous phase having an amine-HCl salt. The acid-washed mixture may be decanted to separate the organic phase and the aqueous phase. The aqueous phase may be neutralized with a base and distilled to produce a recovered amine base additive product, and the organic phase may be distilled to produce an organic solvent distillate product and an MF bottoms product. The latter can be distilled with a heavy solvent to produce a pure MF distillate product and a bottoms product containing OSMs.

As described herein, the selection of an amine base additive and how the amine is added to the process significantly impacts several aspects of the process: (i) the solubility of the amine and its derivatives, which is important to avoid solids formation that plug the reactor, (ii) the ability to recover and re-use the amine, and (iii) the ability to separate the amine from the MF product. The amine base additive in some embodiments preferably reacts quickly with HCl to form a neutral amine-HCl salt. The amine base additive in some embodiments preferably reacts slowly with CMF to form the amine-CMF salt. In some embodiments, the amine base additive and the corresponding amine-HCl salt are soluble in the reaction mixture. The amine base additive in some embodiments preferably has a boiling point greater than the temperature at which the corresponding amine-HCl salt thermally cracks. In preferred embodiments, the amine base additive satisfies one or more, and most preferably all, of these criteria.

The present approach also provides for the production of MFA from ME. In such embodiments, MF is reduced in a solvent in the presence of hydrogen and a catalyst. A suitable catalyst may be used. Preferred catalysts include, Cu, Pd/C, and Pd/Al₂O₃ catalysts. A tertiary amine may be incorporated for extending catalyst life and improving selectivity. It should be appreciated that MFA is an important intermediary for other chemicals, and thus some embodiments will conclude with MFA production. It should also be appreciated that the conversion of MF to MFA may follow from the conversion of CMF to MF.

The present approach also provides for the production of DMF from MF or MFA. In these embodiments, the reactant (MF or MFA) is reduced in a solvent in the presence of hydrogen and a catalyst. A suitable catalyst may be used. Preferred catalysts include Cu, Pd/C, and Pd/Al₂O₃ catalysts. As described herein, a tertiary amine may be selected for extending catalyst life. The amine base additive may be selected in connection with the solvent, as explained below. It should be appreciated that DMF is an important intermediary for other chemicals, and thus some embodiments may conclude with DMF production.

However, due to the advantages of the present approach, some embodiments continue with the production of pX from DMF. In such embodiments, the first step is the reduction of the initial reactant (MF or MFA) in a solvent in the presence of hydrogen and a catalyst to produce DMF. In the second step, the DMF is reacted with ethylene in an organic solvent in the presence of an acid catalyst to produce pX. In some embodiments, the same organic solvent is used for both the production of DMF and the production of pX. This advantageously avoids the need to separate DMF from an initial solvent prior to producing pX from DMF.

These and other embodiments will be apparent to the person having an ordinary level of skill in the art, in view of the following detailed description, drawings, and claims appended hereto.

### DESCRIPTION OF THE DRAWINGS

FIG. 1 illustrates a process for converting CMF to MF according to one embodiment of the present approach.

FIG. 2 illustrates an example of decanting MF and amine base additive according to an embodiment of the present approach.

FIG. 3 shows the estimated time to 99% CMF conversion vs. catalyst loading for 40 wt % and 20 wt % CMF feeds.

FIG. 4 shows MF yield vs. catalyst loading at full CMF conversion.

FIG. 5 shows the molar yield of MF hydrogenation as a function of time for an embodiment of the present approach.

FIG. 6 shows MF, MFA, and DMF yield over time at 230° C.

FIGS. 7 and 8 show molar selectivity of MF hydrogenation at 210° C. and 230° C.

FIG. 9 shows molar selectivity over various reaction temperatures.

FIG. 10 shows molar selectivity as a function of hydrogen pressure in the production of DMF according to the present approach.

FIG. 11 shows molar selectivity for different solvents in the production of pX.

FIGS. 12 and 13 show molar selectivity in DMF production for different concentrations of TBA, at 170° C. and 230° C., respectively.

FIGS. 14 and 15 show molar selectivity as a function of time, with and without TBA as the amine, respectively.

FIGS. 16-18 show flow diagrams for embodiments of the present approach.

### DESCRIPTION

The following description illustrates embodiments of the present approach in sufficient detail to enable practice of the present approach. Although the present approach is described with reference to these specific embodiments, it should be appreciated that the present approach can be embodied in different forms, and this description should not be construed as limiting any appended claims to the specific embodiments set forth herein. Rather, these embodiments are provided so that this disclosure will be thorough and complete, and will fully convey the scope of the present approach to those skilled in the art.

The present approach relates to processes for converting CMF into MF and MFA, MF into MFA, and CMF, MF, and/or MFA into DMF and optionally into pX. The reactions may proceed as batch reactions or continuous reactions.

The conversion of CMF to MF is accomplished by combining CMF with hydrogen in the presence of a supported palladium catalyst. Reaction [I], below, proceeds at mild temperatures and pressures. It should be appreciated that the reaction can continue to produce MFA, if desired.

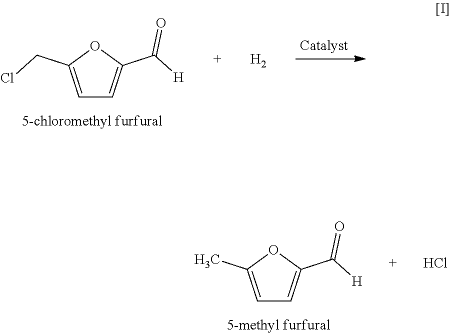

The present approach also relates to the production of 2,5-dimethylfuran (DMF) or p-xylene (pX), from either 5-methylfurfural (MF) or 5-methylfurfuryl alcohol (MFA). The reaction is illustrated below as Reaction [II]. It should be appreciated that DMF may be produced from CMF through first converting CMF to MF and/or MFA.

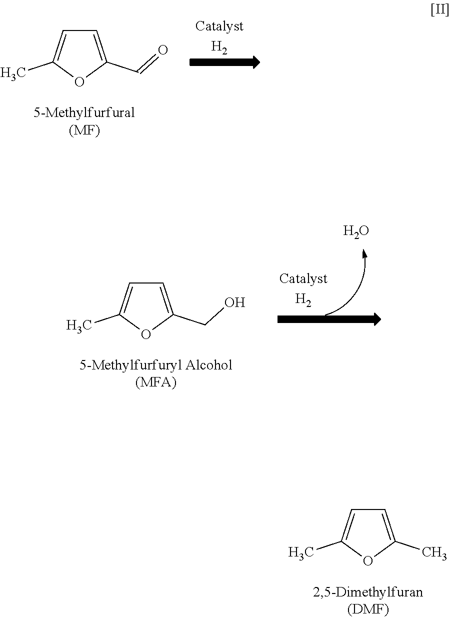

Contemporary processes for converting CMF, such as U.S. Pat. No. 4,335,049 discussed above, suffer from low MF and MFA selectivity and yield, lower catalyst life (e.g., turnover numbers of 1,000 and lower), and form insoluble quaternary salts at typical reaction temperature and pressure conditions. Other contemporary processes, such as those described in U.S. Pat. No. 9,556,137, granted Jan. 31, 2017, and U.S. Pat. No. 10,618,880, granted Apr. 14, 2020, both of which are incorporated by reference in their entirety, are less efficient and require various additional reactants. For example, the aforementioned disclosures call for amides and HCl, respectively, for converting MF to DMF. The present approach does not require such additional components. Further, the tertiary amine discussed below does not form an amide, and hydrogenation is achieved through catalyst use alone in the present approach. The previously mentioned prior art processes used HCl to facilitate the reaction with a Pd catalyst.

The present approach for converting CMF overcomes these deficiencies. In embodiments for converting CMF according to the present approach, a CMF stock in an organic solvent (e.g., toluene) is reacted with an amine base additive in the presence of hydrogen to produce an effluent having MF, the amine base additive, and at least one amine-HCl salt. The reaction proceeds over a Pd catalyst. In some embodiments, the amine-HCl salts are neutralized to produce an MF and amine base additive composition which can be decanted to produce an MF-rich phase and an amine-rich phase. The amine-HCl salt may be neutralized through the use of a caustic agent as discussed below. In such embodiments, the amine-rich phase can be distilled to produce a purified amine base additive and an amine-MF distillate, the latter of which may be recycled to the decanting process. Also, the MF-rich phase may be distilled to produce a purified MF and an amine-MF distillate, the latter of which may be recycled to the decanting process. It should be appreciated that the CMF to MF reaction may proceed in one or more batch reactors, and/or one or more continuous reactors. In the present approach, a reactor may be a back-fed reactor.

The present approach involves the use of an amine base additive in the process. The amine base additive in some embodiments preferably reacts quickly with HCl to form a neutral amine-HCl salt. The amine base additive in some embodiments preferably reacts slowly with CMF to form the amine-CMF salt. In some embodiments, the amine base additive and the corresponding amine-HCl salt are soluble in the reaction mixture. The amine base additive in some embodiments preferably has a boiling point greater than the temperature at which the corresponding amine-HCl salt thermally cracks. In preferred embodiments, the amine base additive satisfies one or more, and most preferably all, of these criteria.

In some embodiments, the amine base additive is a tertiary amine having the formula R₃N, wherein the average carbon length of the R-group is 2-8. Tri-n-butyl amine (TBA), also known as N,N-dibutylbutan-1-amine, is one example of a preferred amine base additive under the present approach. In some embodiments, the amine base additive is a tertiary amine having the formula R₃N, wherein at least one R-group is a branched alkyl, preferably branched at the second carbon from the carbon-nitrogen bond. Tri-isobutyl amine (TiBA), also known as 2-methyl-N,N-bis(2-methylpropyl)propan-1-amine, is another example of a preferred amine base additive under the present approach

Under the present approach, the amine base additive is a tertiary amine of the formula R₃N, wherein each R may be the same or different and is a straight chain alkyl or a branched chain alkyl, and preferably wherein the average number of carbon atoms in each R is from 1 carbon atoms to 12 carbon atoms. Shorter chain lengths and primary or secondary amines tend to react more quickly with CMF to form the undesired and generally insoluble amine-CMF salts. Longer chain lengths (e.g., more than 8 carbon atoms) generally are not as soluble in the reaction mixtures, though there are exceptions. For example, the amine base additive may be one or more of: tri-n-butyl amine (“TBA”), tri-isobutyl amine (“TiBA”), tris-2-ethylhexyl amine (“TEHA”), trihexylamine (“THEX”), dimethyldocecylamine (“DIMLA”), dibutylaniline (“DBAN”), trimethylamine (“TMA”), N, N-diisopropylethylamine (“DJPEA”), tri-n-amylamine, tri-octylamine, and triethylamine (“TEA”).

The following tables provide data and experimental measurements of the behavior of various amines and their salts pertinent to the present approach.

TABLE 1
Solubility of amine and their hydrochlorides in solvents at room temperature.

| | | Solubility of amine hydrochloride salts in different | | | | | |
|---|---|---|---|---|---|---|---|
| | | solvents* (values in wt. %) | | | | | |
| | | | | | methylfurfural | propylene | |
| amine | | toluene | acetone | methanol | (MF) | carbonate | water |
| precursor | solvent | μ = 2.4 | μ = 20.7 | μ = 32.7 | μ = 41.9 | μ = 64.9 | μ = 78.4 |
| trimethylamine | TMA | <0.12 | <0.09 | 34.66 | <0.13 | <1.16 | 38.39 |
| triethylamine | TEA | <0.05 | <0.13 | 26.14 | <0.59 | <0.42 | 34.57 |
| Hunig's base | HB | <4.91 | 15.01 | 33.08 | 26.83 | 34.05 | 33.39 |
| tributylamine | TBA | 29.05 | 35.84 | 38.12 | 34.74 | 29.80 | 36.48 |
| triisobutylamine | TiBA | 51.82 | 34.35 | 47.15 | 24.44 | 31.21 | 25.88 |
| triamylamine | TAA | — | — | — | — | — | — |
| | | Solubility of amines in different | | | | | |
| | | solvents*+ (values in wt. %) | | | | | |
| | | | | | methylfurfural | propylene | |
| | | toluene | acetone | methanol | (MF) | carbonate | water |
| amine | solvent | μ = 2.4 | μ = 20.7 | μ = 32.7 | μ = 41.9 | μ = 64.9 | μ = 78.4 |
| trimethylamine | TMA | — | — | — | — | — | 47.16 |
| triethylamine | TEA | 46.05 | 48.66 | 48.15 | 39.67 | 8.97 | 10.13 |
| Hunig's base | HB | 46.90 | 49.20 | 49.11 | 14.35 | 4.59 | 0.30 |
| tributylamine | TBA | 47.58 | 49.65 | 49.94 | 4.09 | 0.59 | 0.20 |
| triisobutylamine | TiBA | — | — | — | — | — | — |
| triamylamine | TAA | 47.61 | 49.87 | 9.27 | 2.49 | 0.17 | 0.00 |

*For high solubility amines and salts (cells -with bold text), measurements are not necessarily at solute saturation, i.e., not the maximum concentration of amine or salt in the solution
+For low/negligible solubility amines (cells with italics text), measurements are at solute saturation, i.e., at the maximum amine concentration in the solution
Low/negligible (i.e., <15 wt %) High Solubility
μ = dielectric constant

TABLE 2
Cross-solubility of amines and their hydrochloride salts.

| | | Cross-solubility of amines and their hydrochloride salts | | | | | |
|---|---|---|---|---|---|---|---|
| | | | | | methylfurfural | propylene | |
| amine | | toluene | acetone | methanol | (MF) | carbonate | water |
| precursor | solvent | μ = 2.4 | μ = 20.7 | μ = 32.7 | μ = 41.9 | μ = 64.9 | μ = 78.4 |
| trimethylamine | TMA | X | X | — | X | X | V |
| triethylamine | TEA | X | X | V | X | X | X |
| Hunig's base | HB | X | V | V | X | X | X |
| tributylamine | TBA | V | V | V | X | X | X |
| triisobutylamine | TiBA | V | V | V | X | X | X |
| triamylamine | TAA | V | V | X | X | X | X |

Either amine or hydrochloride salt is not soluble
Both amine and hydrochloride salt is soluble

Some embodiments involve a CMF stock that contains organic soluble materials (“OSMs”). In some embodiments the OSMs remain in a solution with MF after separation of the organic solvent and the amine base additive. The MF and the OSMs may be separated using a heavy solvent to prevent or minimize OSM solidification.

The organic solvent may be one or more of a polar organic solvent, methanol, acetone, dimethylformamide, MF, tetrahydrofuran, furfural, a non-polar organic solvent, benzene, alkylbenzene, xylene and toluene. The 5-chloromethylfurfural stock preferably comprises a CMF concentration of less than 60 wt %, or less than 50 wt %, or less than 40 wt %, or less than 30 wt %, or less than 20 wt %, or less than 15 wt %, or less than 10 wt %, or less than 5%, with these concentrations being ±1 wt %. In embodiments of the present approach, the reaction generally takes place at a reaction temperature of 50° C. to 150° C., and for some embodiments more preferably 90° C. to 130° C., and for some embodiments more preferably 100° C. to 120° C., with these temperatures being ±3° C. In embodiments of the present approach, the reaction generally takes place at a hydrogen pressure of 115-515 psia, and for some embodiments more preferably 200-400 psia, and for some embodiments more preferably 215-315 psia, with these pressures being ±5 psia.

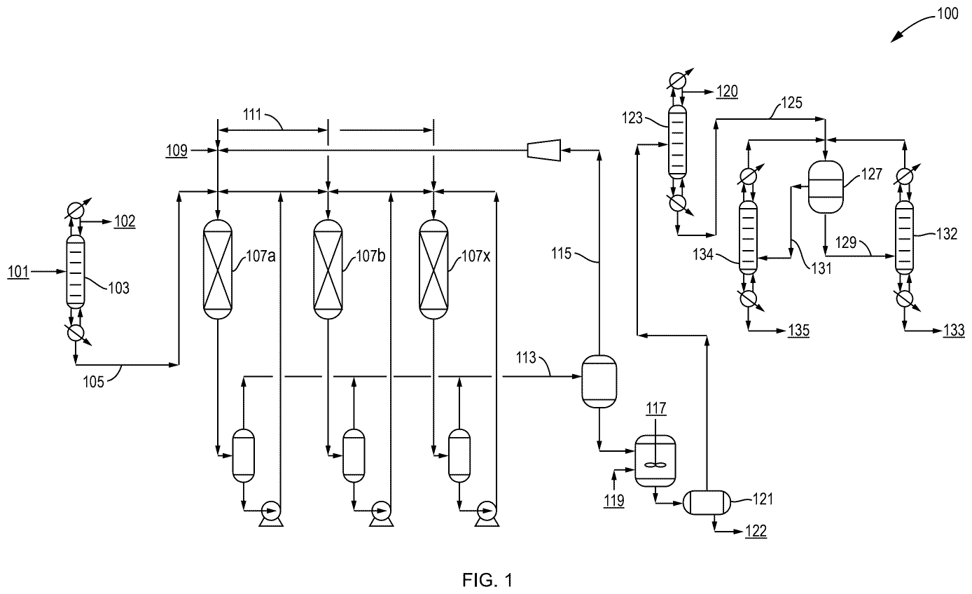

FIG. 1 illustrates a process 100 for converting CMF to MF according to one embodiment of the present approach. The reaction is a hydrodechlorination of CMF over a Pd catalyst in the presence of a tertiary amine base, forming MF and an amine-HCl salt. The CMF is diluted with an organic solvent to a concentration of less than 50 wt %. At least one equivalent of the amine base additive is added relative to CMF. The drawing employs standard symbols used in the chemical engineering arts. An organic effluent 101 containing CMF and an organic solvent (e.g., toluene), enters a first distillation column 103. The organic solvent 102 may be recovered and recycled, and the concentrated CMF 105 exits and enters a series of reactors 107a-107x, preferably continuously stirred tank reactors (“CSTRs”), in the presence of hydrogen 109 and a tertiary amine 111, over a Pd catalyst. In some embodiments, at least one equivalent of the amine base additive 111 may be added in stages over the course of the reaction. Other reactors may be used, but CSTRs advantageously limit or prevent precipitation or amine-CMF quaternary salts. The CMF is preferably diluted with an organic solvent to less than 60 wt %, and in some embodiments less than 50 wt %, and in some embodiments less than 40 wt %, and in some embodiments less than 30 wt %, and in some embodiments less than 20 wt %, and in some embodiments less than 15 wt %, and in some embodiments less than 10 wt %, and in some embodiments less than 5%, with these concentrations being ±1 wt %. The CSTRs produce an effluent 113 of MF, organic solvent, amine base additive, amine-HCl salt, and OSMs. Effluent 113 may be separated to remove hydrogen for hydrogen recycle 115.

The initial amine base additive 111 may be recovered from the amine-HCl salt by neutralizing 117 the amine-HCl salt with an aqueous base 119, forming the amine and an aqueous chloride salt, where the amine is separated from the aqueous phase by decantation 121, removing waste water 122. The reaction may be performed in a series (two or more) back-mixed reactors.

Following the reaction, the composition 113 includes MF, organic solvent (e.g., toluene), amine base additive (e.g., TBA), amine-HCl salts, and OSMs. Advantageously, the OSMs formed from biomass conversion products do not need to be separated at this stage and may remain in the solution until the separation and purification of MF.

The organic solvent 120 may be separated from the organic phase effluent by distillation 123, leaving a stream 125 comprising MF and amine for phase separation 127, where the MF and amine forms separate MF-rich phase 192 and amine-rich phase 131, which are then separately purified into MF 133 and amine 135 by a distillation processes 132 and 134, respectively.

The organic solvent 120 may be a single compound, as discussed above. In some embodiments, however, the diluent/solvent in the reaction comprises the combination of a non-polar solvent (such as toluene) and a polar solvent such as methanol, acetone, dimethylformamide, MF, tetrahydrofuran, benzene, alkylbenzene, xylene or furfural.

As shown in FIG. 1, the unreacted amine in the reactor effluent 113 may be reacted with aqueous HCl such that essentially all amine is converted to amine-HCl. The aqueous phase (containing amine-HCl) is then separated from the organic phase by decantation. The aqueous phase is neutralized with a base to recover the amine from the aqueous chloride salt by decantation.

It should be appreciated that the process illustrated in FIG. 1 is configured for a process involving TBA as the amine base additive. The chemistry may change for using other amine base additives, requiring variations in the processing.

The present approach may feature one or more separation operations following the CMF to MF reaction, and in some embodiments after amine-HCl salt neutralization. For example, a first separation may remove organic solvent, leaving MF, the amine base additive, and any OSMs for subsequent separation operations. MF and amine base additives may be separated through decanting to produce amine-rich and MF-rich phases. Those phases, in turn, may be distilled to produce pure bottoms products, and the distillate products may be returned to the decanting operation.

In some embodiments, the reactor effluent is mixed washed with dilute HCl to produce an acid-washed mixture of an organic phase having MF and the organic solvent, and an aqueous phase having an amine-HCl salt. The acid-washed mixture may be decanted to separate the organic phase and the aqueous phase. The aqueous phase may be neutralized with a base and distilled to produce a recovered amine base additive product, and the organic phase may be distilled to produce an organic solvent distillate product and an MF bottoms product. The latter can be distilled with a heavy solvent to produce a pure MF distillate product and a bottoms product containing OSMs.

It should be appreciated that the present approach can involve various combinations of separations processes depending on the particular amine base additive. For example, in some embodiments the process may involve:
reacting a CMF stock having CMF, OSMs, and an organic solvent, with an amine base additive in the presence of hydrogen to produce an effluent having MF, OSMs, the organic solvent, any remaining amine base additive, and at least one amine-HCl salt;
neutralizing the effluent with an aqueous base to produce a neutralized mixture of MF, OSMs, the organic solvent, and neutralized amine;
distilling the neutralized mixture to produce an organic solvent distillate product and a bottoms product having MF, OSMs, and the neutralized amine;
decanting the bottoms product to produce an amine-rich phase having the neutralized amine, and an MF-rich phase having MF and OSMs;
distilling the amine-rich phase to produce a recovered amine; and
distilling the MF-rich phase to produce a recovered MF product.

As another example, some embodiments of the present approach may involve a thermal cracking operation followed by a different sequence of separations. For example, some embodiments may involve:
reacting a CMF stock having CMF, OSMs, and an organic solvent, with an amine base additive in the presence of hydrogen to produce an effluent having MF, OSMs, the organic solvent, any remaining amine base additive, and at least one amine-HCl salt;
thermally cracking the effluent to produce a recovered HCl product, an organic solvent distillate product, and an MF-rich bottoms product having MF, an amine, and OSMs;
distilling the MF-rich bottoms product to produce an MF distillate product and an amine-rich bottoms product containing the amine and the OSMs;
distilling the amine-rich bottoms product with a base to produce an amine distillate produce and an OSMs bottoms product.
   Such embodiments may include, for instance, toluene as the organic solvent is toluene and TBA or TEHA as the amine base additive.

As yet another example, some embodiments may involve separations operations to remove OSMs from other components. For example, some embodiments may involve:
preparing a CMF slurry having CMF, OSMs, an amine base additive, and an organic solvent;
mixing the CMF slurry with an organic alcohol to produce a homogenous CMF stock having CMF, the amine base product, the organic solvent, quaternary salts, and the OSMs;
reacting the homogenous CMF stock in the presence of hydrogen over a Pd catalyst to produce an effluent having MF, OSMs, the organic solvent, any remaining amine, at least one amine-HCl salt, and methanol;
neutralizing the effluent with an aqueous base to produce a neutralized effluent of MF, OSMs, the organic solvent, methanol, and a neutralized amine;
distilling the neutralized effluent to produce an amine distillate product and an organic-rich bottoms product having MF, OSMs, the organic solvent, and methanol;
distilling the organic-rich bottoms product to produce a methanol and organic solvent distillate product and an MF-rich bottoms product having MF, OSMs, and residual organic solvent;
distilling the MF-rich bottoms product to produce a residual organic solvent distillate product and an MF bottoms product having MF, OSMs; and
distilling the MF bottoms product in the presence of a heavy solvent to produce an MF distillate and an OSMs bottoms product.
   In such embodiments, the methanol and organic solvent distillate product may be recycled to the homogenous CMF stock. As will be evident to the person having an ordinary level of skill in the art, embodiments may feature amine base additive recovery operations and separations operations other than as described in the demonstrative embodiments. Further, the amine base additive recovery operations and separations operations may depend on the specific organic solvent and/or amine base additive used.

It should be appreciated that embodiments for converting CMF according to the present approach allow for exceptionally favorable turnover numbers (“TON,” a ratio of the number of moles of a product produced by a mole of catalyst before the catalyst becomes inactivated). Table 3, below, shows data for converting CMF to MF according to embodiments of the present approach (Runs 1-16) and contemporary processes (Runs 17-24). The data includes the solvent and amine selection, amine ratio relative to CMF, CMF turnover number (mol CMF/mol Pd catalyst), hydrogen pressure, reaction temperature and time, CMF conversion, MF yield, and MF turnover number. Runs 17-19 are data reported in U.S. Pat. No. 4,335,049, and Runs 20-24 used the solvent and amine described in U.S. Pat. No. 4,335,049 at different loads and conditions. As can be seen, Runs 1-16 operated at CMF TONs of 7,000 to over 145,000, MF TONs of 5,000 to over 100,000, high CMF conversion, and high MF yields. In comparison, Runs 17-19 had exceptionally low TONs of 20-22, and therefore consumed catalyst at an unacceptable rate. Likewise, Runs 20-24 shows that the present approach is superior to contemporary processes. For example, Run 20 achieved an MF yield of only 61% with a, and Run 21 required 22 hours to achieve an MF yield of 91% but a CMF TON of only 200. The CMF TONs increased to 1,000-5,000 for Runs 22-24, but the MF yields decreased dramatically (8%, 75%, 47%, respectively). These data demonstrate the superior performance of the present approach for converting CMF.

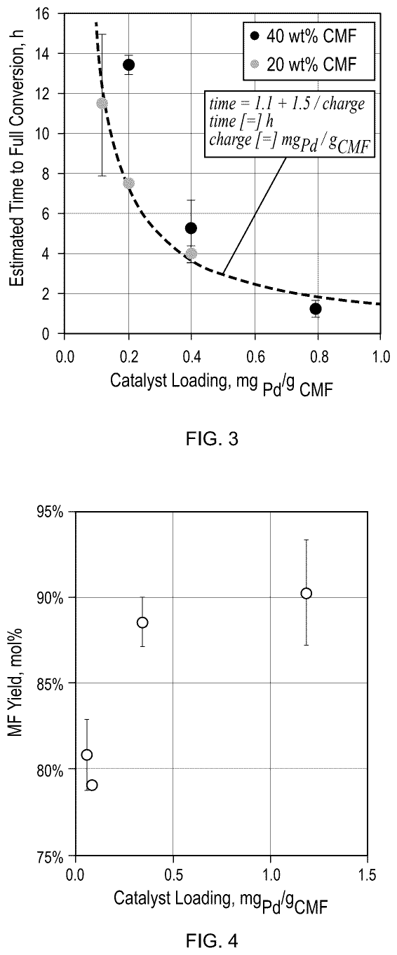

In the present approach, increasing the catalyst-to-CMF ratio lowers reactions times and increases MF yields. As seen in FIG. 3, increasing the catalyst loading (expressed in mg Pd/g CMF) decreases the estimated time (expressed in hours) to achieving a 99% conversion. FIG. 4 shows MF yield (in mol %) as a function of catalyst loading (expressed in mg Pd/g CMF) for 99% CMF conversion.

Some embodiments of the present approach for converting CMF may neutralize amine salts formed during the reaction. After the reaction, the amine-HCl salt (e.g., tributylamine hydrochloride in some embodiments) may be neutralized with an aqueous base or inorganic base to recover the tertiary amine for reuse. Amine recovery dramatically increases the economic feasibility of the process, at least because amines are a high-value chemical. Inorganic bases that have been evaluated and found effective for the neutralization of the hydrochloride salt include sodium hydroxide (NaOH), sodium bicarbonate (NaHCO₃), magnesium hydroxide (Mg(OH)₂), calcium oxide (CaO), and calcium hydroxide (Ca(OH)₂). For neutralization, a molar excess of base may be used to ensure complete neutralization and account for any chlorinated OSMs present. A higher ratio of base to CMF results in a higher recovery of amine, because the excess base reacts with amine-HCl salt to release free amine. In some embodiments, a 100%-200% base to amine ratio is suitable for maximizing amine recovery, including, e.g., 110%, 120%, 130%, 140%, 150%, 160%, 170%, 180%, 190%, and 1% increments therebetween.

Strong bases, such as NaOH, can react with the carbonyl group in MF through nucleophilic attack. This reaction leads to a decrease in MF yields by forming products that may be undesirable, such as 5-methylfurfuryl alcohol (MFA) and 5-methylfuroic acid (MFCA). Moreover, hydroxide bases may catalyze polymerization reactions, resulting in the formation of oligomer species that precipitate as solids during the washing process.

On the other hand, Ca(OH)₂ neutralization advantageously provides better recoveries of the desired components, MF and amine, especially compared to NaOH neutralization. Note that these bases are not soluble in water or the organic phase, so they do not require an aqueous phase but are added as solids to the organic effluent.

TABLE 2
MF and amine recovery after amine-HCl salt neutralization.

| Neutralizing base | MF recovery | Amine recovery |
|---|---|---|
| Ca(OH)₂ powder | 96% | 91% |
| Ca(OH)₂ powder | 95% | 77% |
| CaO | 94% | 67% |
| Mg(OH)₂ | 73% | 24% |

In a second process, unreacted amine in the reactor effluent may be titrated with aqueous HCl to convert all amine to the amine-HCl salt. The amine-HCl salt is soluble in the aqueous phase and can be separated from the organic phase by decantation. The aqueous phase is then neutralized with a base, “springing’ the amine which can be separated by decantation. This process avoids having to recover amine from the organic effluent by distillation.

In some embodiments of the present approach, formation of insoluble amine-CMF salts cannot be completely avoided when using tertiary amines with 3-5 carbon chain length (average of all R groups). However, under the present approach the rate of formation can be further minimized by practicing one or both of the following techniques.

The first technique involves staged injection of the amine, minimizing the amount of excess amine relative to CMF throughout the reactor. The overall amount of amine must be at least equal to one equivalent of amine per CMF in the feed, but the ratio of amine to unreacted CMF can be minimized by staged injection as the reaction proceeds. This technique reduces the reaction rate of amine with CMF to form insoluble amine-CMF salts which tend to plug the reactor.

In a second technique involves back-mixing of reactor effluent with reactor feed. This approach reduces the CMF concentration in the reactor. Preferably the reactor is a perfectly back-mixed reactor, or CSTR, in which the CMF concentration throughout the reactor is equal to the exit concentration, which is lower than the feed concentration. Minimizing CMF concentration reduces the rate of reaction with amine, forming insoluble amine-CMF salts that tend to plug the reactor. In order to achieve high overall conversion of CMF, several back-mixed reactors in series can be used. In some embodiments, 2-6 back-mixed reactors can be used. In a more preferred embodiment, 3 back-mixed reactors can be used.

A diluent (solvent) has been found to also help reduce reactions of CMF that lead to solids formation. In some embodiments, the CMF concentration is maintained below about 60 wt %, and preferably below about 50 wt %, and more preferably below about 30 wt %. Non-polar solvents such as toluene serve as effective diluents. Polar solvents, such as methanol, acetone, dimethylformamide, MF, tetrahydrofuran and furfural may also be effective, as both a diluent for CMF and as a solvent for some of the degradation products that might otherwise precipitate in non-polar solvents. Thus, polar solvents, and optionally polar solvents in conjunction with non-polar solvents, can lead to longer reactor run lengths without plugging from solids.

Another feature of the present approach involves separation of the MF product from the amine. In contemporary processes, it is very difficult to separate amines such as TBA from MF by distillation. There is an azeotrope between MF and TBA containing 60-70 wt % MF. At temperatures below about 60° C., this azeotropic composition phase separates, with an MF-rich phase on the bottom and a TBA-rich phase on top. These phases can be decanted and distilled separately form relatively pure MF and pure TBA.

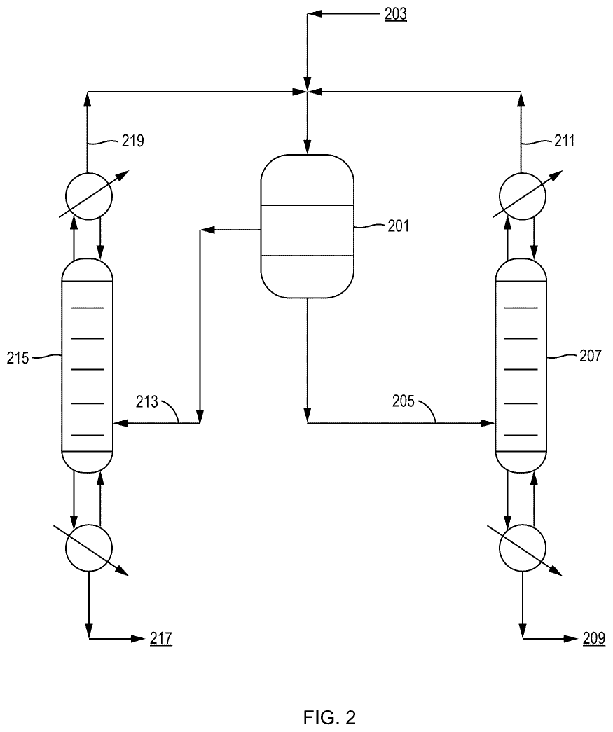

FIG. 2 illustrates an example of decanting MF and amine base additive according to an embodiment of the present approach. As shown, decanter 201 is fed a composition 203 containing MF and amine base additive. The MF-rich phase 205 exits decanter 201 into a first distillation column 207 producing pure (e.g., 99%) MF 209 with the top product 211 returned to decanter 201. The TBA-rich phase 213 exits decanter 201 into a second distillation column 215, producing pure (e.g., 99%) TBA 217 with the top product 219 returned to decanter 201. Embodiments of the present approach may take the form of a process for separating MF and TBA. Some embodiments may include a process for separating MF and TBA as a component of a process, such as converting CMF to MF.

Another aspect of the present approach relates to the preparation and purification of the CMF feedstock. When CMF is produced by the hydrolysis of cellulosic feedstocks using HCl and chloride salts in a biphasic reactor with an organic solvent, small quantities of other byproducts are produced in the organic phase. These byproducts are referred to as Organic Soluble Materials, or OSMs. The OSMs tend to be unstable and separation of CMF from the OSMs can be extremely difficult. Advantageously, under the present approach the CMF does not need to be separated from OSMs prior to the hydrodechlorination reaction that converts CMF to MF. As an additional benefit, the OSMs undergo some degree of hydrogenation and dechlorination, improving their stability and value as renewable fuels and other products.

In the present approach, the dechlorination reaction to convert CMF to MF can be performed in either a fixed bed reactor or a continuous stirred tank reactor (“CSTR”) with a noble metal catalyst. In preferred embodiments of this reaction, an amine base additive is added to scavenge HCl which enhances the catalyst life. The following paragraphs describe variations that may be included in the reaction step.

Some embodiments may include a staged amine injection. In these embodiments, an amine base additive is injected in a staged manner to prevent amine-CMF quat salt formation.

Some embodiments may include recirculating a fraction of the product of the CMF to MF reaction. A fraction of the product from CMF to MF reaction step is recycled back to the reactor to reduce the CMF concentration in the incoming feed and thereby slow down the rate of amine-CMF quat salt formation.

In some embodiments, a co-solvent may be added to the incoming composition containing the initial CMF reactant. A co-solvent such as methanol can be used for dissolving CMF-TBA quat salt during CMF to MF reaction.

After the reaction, the CMF to MF conversion product contains the organic solvent (e.g., toluene), MF, any remaining amine base additive, amine-CMF quaternary salt, amine-HCl salt, OSMs-amine quaternary salt, and OSMs. The proportion of these components will vary depending on the specific embodiment. Following the reaction, embodiments of the present approach may include one or more separation processes to separate and recover various components of the conversion product, such as MF, HCl, amine, and OSMs. The following paragraphs describe various aspects of the separation processes that may be included in embodiments of the present approach for converting CMF.

If TEHA is used as the amine base additive, then HCl can be recovered via a process such as thermal cracking. Otherwise, the amine-HCl salt may be neutralized with a caustic material to free the amine from the amine-HCl salt. The HCl is lost in the form of NaCl during the neutralization step.

After a caustic wash, the organic phase may be distilled to obtain solvent (e.g., toluene), MF, amine base additive, and OSMs. The distillation sequence may be designed based on the following criteria. First, it is recommended to vaporize light material only once to reduce both reboiler and condenser duty. Second, high-purity products (e.g., MF) are preferably collected from the overhead. This avoids heavy impurities from the OSMs degradation in the MF product. Third, it is recommended to perform the most difficult separation as a final step in the separation processes. Some embodiments of the present approach follow one or more of these criteria. Preferred embodiments follow all three criteria. Of course, the person having an ordinary level of skill in the art may choose a separation process that contradicts one or more of these criteria without departing from the present approach.

It should be appreciated that the MF and TBA mixture present in some embodiments of the present approach is somewhat unique, at least in the sense that the mixture forms a biphasic mixture. However, the biphasic mixture is not a clear separation. The top layer dominates in TBA and the bottom layer dominates in ME. In addition, the MF and TBA system forms an azeotrope that constrains the MF purity to below 70 wt %. This behavior may be circumvented by exploiting the biphasic nature of the MF and TBA mixture. The phase separation may be performed prior to distillation, producing two high-purity streams. Both the TBA-rich fraction and the MF-rich fraction can be distilled separately to obtain nearly pure TBA and MF products in the respective bottom streams. The overhead from these distillations contains the MF/TBA azeotrope that can be recycled back to the phase separation operation.

Some embodiments may feature an alternative amine recovery operation. In this alternative, amine is recovered using a dilute acid followed by caustic wash as described elsewhere herein. The route avoids complications between MF and amine separation.

As discussed above, certain CMF stock, and in particular crude CMF, contains OSMs. Including a heavy solvent, such as Dowtherm A™ (Dow Chemical Company, Midland, MI) or A200™ (Shell Chemical Co., Houston, TX), can be added during the distillation operations to prevent OSM solidification.

It should be appreciated that a variety of compounds may be suitable as the amine base compound, although some have advantages. Amine evaluation is ongoing, and it should be appreciated that amine compounds not specifically referenced herein may prove effective in embodiments of the present approach. Based on the amines screened for the CMF to MF reaction, tertiary amines with three identical R groups, e.g., tributylamine (TBA) and trihexylamine (THEX), have proven most successful. Tertiary amines in which at least one of the R groups was different, e.g., dimethyldocecylamine (DIMLA) and dibutylaniline (DBAN), have been evaluated and may be effective in some embodiments. DBAN specifically has a phenyl group as one of the R groups. Other aromatic amines, such as aniline, have also been evaluated and can be used. One or more of the R groups may be branched alkyl chains, such as triisobutylamine (TiBA) and tris(2-ethylhexyl)amine (TEHA).

The operations needed for a given embodiment will depend on amine selection. The following paragraphs describe various embodiments of the present approach specific to the amine base compound, and with examples of alternative operations. It should be appreciated that these are merely demonstrative of the present approach, and that modifications to the various operations may be made without departing from the present approach. Unit operations common in chemical engineering are illustrated using conventional symbols.

Some embodiments of the present approach convert CMF to MF using TBA as the amine base compound, and toluene as the solvent, according to an embodiment of the present approach. The initial feed may be a concentrated, purified CMF feed, but may contain OSMs as described above. The hydrodechlorination (“HDCl”) reaction proceeds in fixed bed reactors over Pd catalyst in a hydrogen environment. Following the HDCl reaction, the reaction products include MF, TBA, toluene, TBA-HCl salts, and OSMs. The process continues through hydrogen recovery, acid neutralization using a caustic agent, and solvent recovery, before the decanting operation to separate pure amine and pure MF. Distillate overheads may be recycled as discussed elsewhere herein. It should be noted that “pure,” as used herein, refers to a composition containing at least 80 wt % of the compound, or at least 85 wt % of the compound, or at least 90 wt % of the compound, or at least 95 wt % of the compound, or at least 99 wt % of the compound.

In an alternative embodiment, CMF is converted to MF using TBA in continuous stirred tanks reactors. Hydrogen and the amine are introduced to the CSTRs as illustrated. Otherwise, the process continues as discussed above with respect to the previous embodiment.

In another embodiment, CMF is converted to MF using TBA and a fixed bed. In this embodiment, hydrogen is recovered from the reaction products, and then a dilute acid wash is used to recover the amine. Following the acid wash, the MF-rich component is distilled to separate MF from the organic solvent (e.g., toluene), and the aqueous phase with TBA-HCl salts is neutralized (e.g., via NaOH) and TBA is recovered.

In a variant of the previous process, CSTRs are used for the CMF to MF reaction. Following the CSTR operation, the MF, solvent, and amine may be recovered as described in connection with the previous embodiments.

In another variant, fixed bed reactors are used for the CMF to MF reaction, and a caustic agent is used to neutralize TBA-HCl salts. In this embodiment, the solvent (toluene) is first recovered, and then the process separates amine and MF phases through decantation as discussed above. The phase splitting is effective for overcoming breaking the amine-MF azeotrope. The TBA-rich phase is distilled to produce pure TBA, and the MF-rich phase is distilled to produce MF containing OSMs. Distillation overhead is returned to the decanter as discussed elsewhere. A heavy solvent is used to prevent or minimize OSM solidification during the distillation to separate MF from OSMs.

In a variation of this process, the CMF to MF reaction proceeds in CSTRs. Following the CMF to MR reaction, the amine-HCl salts are neutralized using a caustic agent, and then the solvent is removed to produce an MF-amine product. The MF and amine are separated as described in connection with the previous embodiment.

In another embodiment in which the CMF to MF reaction proceeds in fixed beds, a dilute acid is used prior to amine recovery. In this variant, an acid wash is used to neutralize amine-HCl salts before an amine recovery operation. In this alternative method, amine is recovered using a dilute acid, followed by caustic wash. The aqueous amine-HCl salt phase is neutralized with a base (e.g., NaOH), and then the amine is recovered. The MF and solvent are separated as discussed above, and the MF is separated from OSMs using a heavy solvent as discussed above. It should be appreciated that this approach avoids the complications of separating MF from the amine base additive.

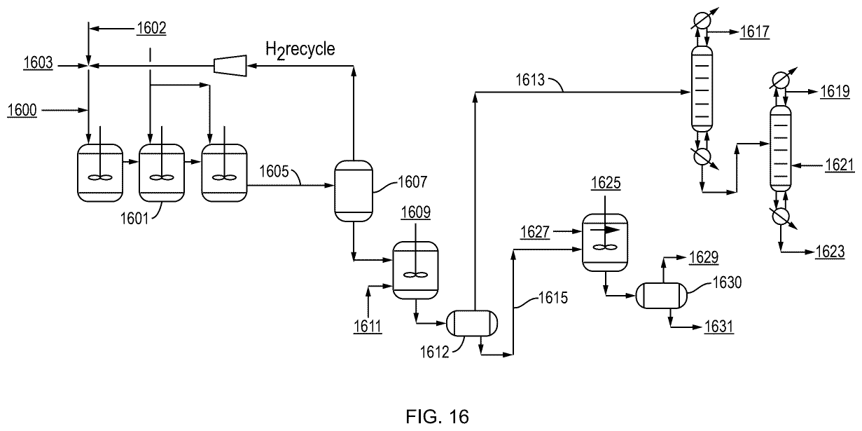

FIG. 16 shows a process flow diagram for an alternative of the previous process. In this embodiment of the present approach, the CMF to MF reaction proceeds with CMF stock 1600 and an amine base additive 1602 fed into CSTRs 1601 in hydrogen 1603 and with a Pd catalyst. Composition 1605 exiting reactors 1601 includes MF, solvent (e.g., toluene), amine (e.g., TBA), amine-HCl salts, and OSMs. Any unused hydrogen may be recycled 1607, prior to an acid wash process 1609 with a dilute acid 1611. Solvent phase 1613 with solvent, MF, and OSMs may be separated 1612 from aqueous phase 1615 having amine-HCl salts. Solvent (e.g., toluene) may be removed from solvent phase 1613 as distillate overhead product 1617, and then MF may be removed as distillate overhead product 1619 with the addition of a heavy solvent 1621 to remove OSMs 1623. Amine may be recovered from aqueous phase 1615 through neutralization 1625 with, e.g., a base such as NaOH 1627, followed by phase separation 1630 to separate amine (e.g., TBA) 1629 and wastewater 1631. In this embodiment, TBA is advantageously removed via acid wash 1609 prior to MF recovery to avoid the amine-MF azeotrope.

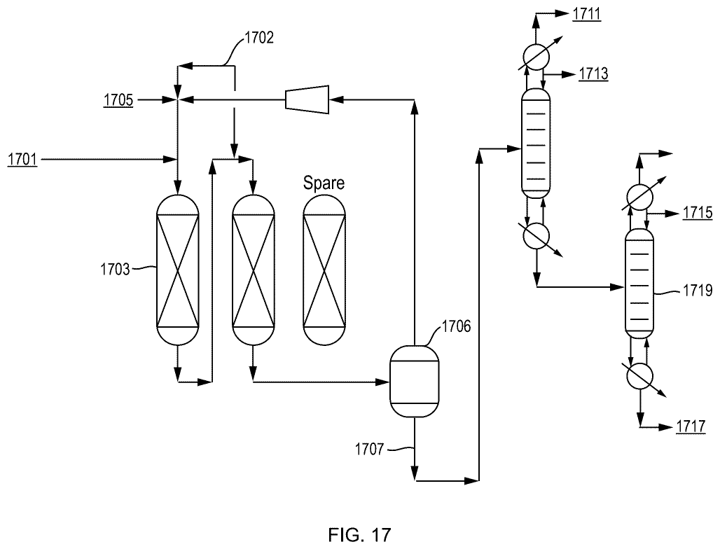

As referenced above, other amines can be used as the amine base compound. The necessary operations may vary to account for the chemistry specific to the amine. FIG. 17 illustrates a process flow diagram for the CMF to MF reaction in which TEHA is the amine base additive. In this embodiment, CMF stock 1701 and amine (e.g., TEHA) 1702 are fed to fixed bed reactors 1703 in hydrogen 1705 over a Pd catalyst. After hydrogen recovery 1706, the reactor effluent 1707 includes MF, solvent, TEHA, TEHA-HCl salt, and OSMs. TEHA can be challenging to recover as an overhead distillate. However, thermal cracking operation 1709 may be used to recover HCl 1711 and solvent (e.g., toluene) 1713, resulting in a bottoms product of MF 1715 and TEHA 1717 that can be separated via distillation 1719. Advantageously, this embodiment avoids the difficulty of recovering TEHA as an overhead product, and also allows HCl recovery through thermal cracking.

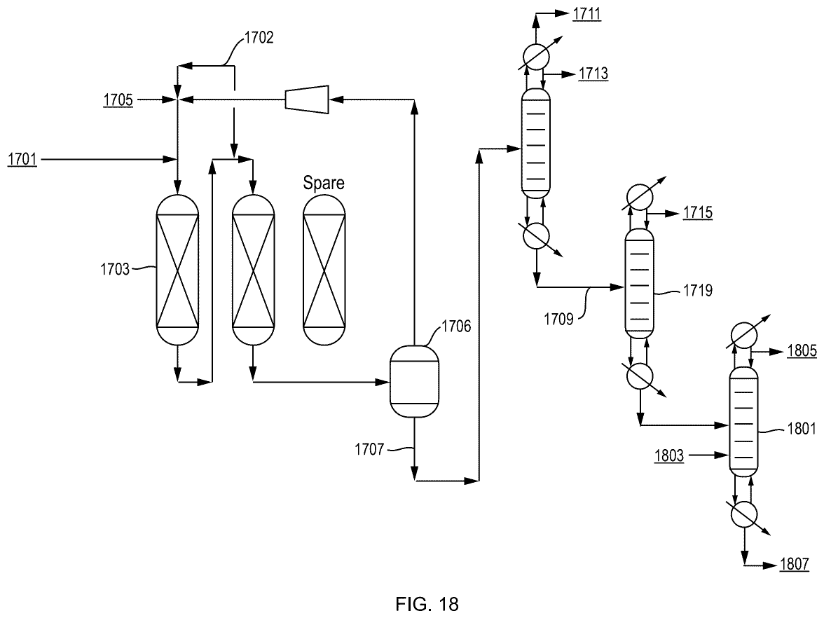

FIG. 18 shows a process flow diagram for a variant of the process in FIG. 17, in which an additional distillation operation 1801 is used to separate the amine from OSMs. Heavy solvent 1803 is used to separate amine (e.g., TEHA) as a distillate overhead product 1805, and OSMs are recovered as a bottoms product 1807. As discussed above, a heavy solvent may be used to prevent or minimize OSM solidification.

Another embodiment has been demonstrated using trimethylamine (“TMA”). In this embodiment, CMF is converted to MF using TMA as the amine base additive. The crude CMF and the amine base additive are fed to quaternary salt reactors to produce a homogenous slurry of CMF, TMA, OSMs, amine salts of CMF and OSMs, and MeOH. It should be appreciated that the MeOH is introduced to a final stage prior to the CMF to MF reaction and is recovered for recycle later in the process. The presence of methanol in the slurry is advantageous because amine adducts, CMF, and OSMs are soluble in methanol. The slurry proceeds to fixed bed reactors for the CMF to MF HDCl reaction over a Pd catalyst in hydrogen. Hydrogen is recycled from the reactor effluent, and the remaining composition enters amine recover, solvent recovery, and MF purification operations. In this embodiment, the salts are neutralized with a caustic agent, springing the TMA for recovery as a distillate overhead. Next, MeOH and toluene from the bottoms product are recovered as overhead distillates, and then a heavy solvent is used to remove OSMs and produce a pure MF overhead distillate.

In a variant of the embodiment using TMA, the MF bottoms product may be recovered without an additional operation to remove OSMs. After neutralization, TMA may be removed as an overhead distillate in a first distillation process, MeOH in toluene may be removed as an overhead distillate in a second distillation process, and finally MF and toluene may be separated in a third distillation process. This illustrates an advantage of the present approach—individual operations may be used as necessary for given embodiment and are incorporated as needed to account for the chemistry of the amine base additive.

In some embodiments, TEA is the amine base additive. The process proceeds through quaternary salt reactors, the CMF to MF reaction in fixed bed reactors, and amine-HCl salt neutralization operations as described above. In this embodiment, however, the solvents (toluene and MeOH) are recovered first as overhead distillates. The amine is next recovered as an overhead distillate, and then MF is recovered and separated from OSMs using the previously described operations. Depending on the embodiment, an additional distillation to remove solvent from MF may be needed prior to removing OSMs.

In a variation of the TEA embodiment, the reactor effluent proceeds through an amine-HCl salt neutralization operations as described above, and then MeOH/toluene are removed and recycled. Next, TEA is recovered, and then the solvent is separated as an overhead distillate to produce a MF bottoms product.

In some embodiments, the present approach takes the form of processes for converting MF and/or MFA to DMF. It should be appreciated that this process may follow the conversion of CMF to MF and/or MFA discussed above. Reaction scheme [II] below illustrates the reaction according to an embodiment of the present approach. MF first reduces to MFA in the presence of hydrogen and a catalyst. The reaction continues with the reduction of MFA to DMF in the presence of hydrogen and a catalyst. Water is the byproduct of the reduction reactions, and may be separated and removed from the reactor(s) using common techniques in the art. It should be appreciated that the same reaction scheme is applicable to embodiments utilizing MFA as the initial reactant.

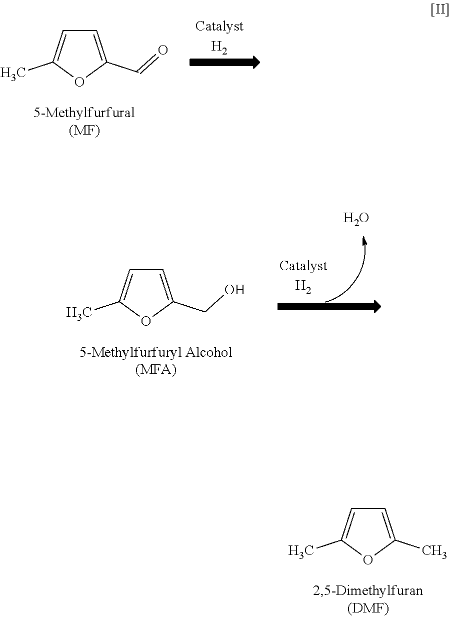

In the reduction of M and MFA into DMF processes, the reactant (MF or MFA) is reduced in a solvent in the presence of hydrogen and a catalyst. Preferred catalysts include Cu, Pd/C, and Pd/Al₂O₃ catalysts. In some embodiments employing a Pd/C or Pd/Al₂O₃ catalyst, a tertiary amine may be incorporated for extending catalyst life as discussed above. It is preferable to select a solvent/amine system as discussed above.

Some embodiments of the present approach are processes to produce p-xylene from 2,5-dimethylfuran. In these processes, DMF is reacted with ethylene in an organic solvent in the presence of an acid catalyst. Reaction scheme [III] below shows the reaction for producing pX from DMF, according to an embodiment of the present approach. As can be seen, DMF reacted with ethylene in the presence of an acid catalyst and an organic solvent to make pX. Water is the byproduct of the reaction, and may be separated and removed from the reactor(s) using common techniques in the art.

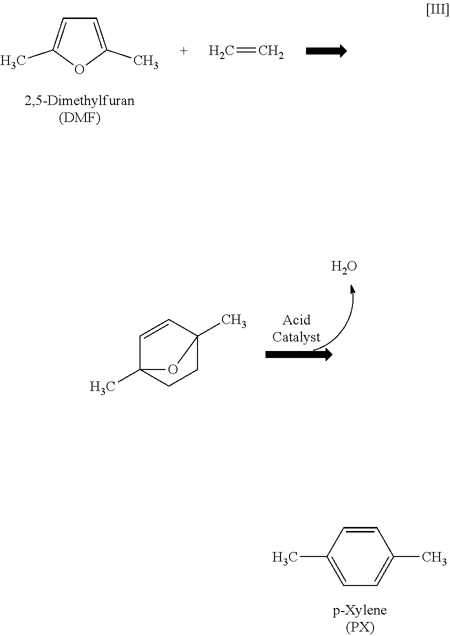

Also described herein are processes for producing p-xylene from 5-methylfurfural. It should be appreciated that these embodiments are a combination of the processes for producing DMF from MF and/or MFA, and for producing pX from DMF. For example, the reaction scheme for these embodiments follows reaction scheme [II] and then reaction scheme [III]. In such embodiments of the present approach, the process first involves reacting 5-methylfurfural with hydrogen over a metal catalyst in an organic solvent. This reaction produces 2,5-dimethylfuran. Embodiments of the process next involve reacting the 2,5-dimethylfuran effluent with ethylene in an acid-catalyzed environment to produce p-xylene. In some embodiments, the 2,5-dimethylfuran effluent from the reduction of 5-methylfurfural is used without separation for producing p-xylene. The p-xylene effluent may then be separated from the solvent and any impurities to produce high-purity p-xylene product. In some embodiments, the solvent may be recycled for use in the reduction of 5-methylfurfural.

As discussed above, the reaction proceeds in a solvent, and preferably an organic solvent. Solvent selection should not have a major impact on the reaction of MF/MFA to DMF in the vapor phase if the solvent is inert to the chemistry. However, the reactions in the stirred tank reactor can result in phase separation with aliphatic solvents. The solubility of MF/MFA in organic solvents is higher when the solvent is aromatic rather than aliphatic.

Examples of preferred organic solvents are toluene, ethylbenzene, p-xylene, mixed xylenes, tetralin, naphthalene, methylnaphthalene, C9 to C18 aromatic solvents, linear or branched paraffin in the C8 to C20 carbon range, and p-diethylbenzene. It should be appreciated that combinations of more than one solvent may be used, and that other solvents may be used. Further, different solvents may be used for the reduction of MF and MFA, and the conversion of DMF to pX, although it may be necessary to separate the solvent from the initial reaction before the second reaction.

The following observations were made during demonstrative runs of converting MF to DMF according to the present approach:
Liquid-liquid separation is observed using both dodecane and Parafol-14 as the solvent.
Phase separation results in lower observed reaction rates.
When phase splitting is observed, the heavy organic phase has a high amount of 2-5 hexanedione and 2,5-hexanediol. In the presence of acidic sites and water, 2,5-hexanedione is in equilibrium with DMF and can be leveraged to recover DMF. However, further hydrogenation of 2,5-hexandione will produce 2,5-hexanediol and cannot be converted back into DMF as this species is not in equilibrium with DMF.
Lower reaction rates may be explained by mass transfer effects due to heterogeneity.
Experiments with aromatic solvents such as toluene have shown high MF conversion and DMF selectivity in a stirred tank reactor.

The initial concentration of MF and/or MFA in the solvent is preferably between 5-70 wt %, but it should be appreciated that the concentration may vary depending on the embodiment. For example, the concentration may be 10 wt %, 20 wt %, 30 wt %, 40 wt % 50 wt %, 60 wt %, or any 1 wt % increment between those values.

As discussed above, the reduction reactions of MF and MFA proceed with a catalyst. In preferred embodiments, the catalyst is a transition metal catalyst, and more preferably a noble metal catalyst. The catalyst may include acid sites. As described in the demonstrative embodiments, three different catalysts have been demonstrated. These catalysts are bulk Cu catalyst, Pd/C, and Pd/Al₂O₃. Both Cu and Pd/Al₂O₃ showed good reactivity relative to Pd/C in the demonstrative embodiments. Greater selectivity has been demonstrated for Cu compared to Pd/C and Pd/Al₂O₃, and the Pd catalysts appear to favor ring hydrogenation and opening chemistry. The chemistry is not exclusive to the two catalysts mentioned above as other transition metal catalysts can be effective for the chemistry, with group 9, 10, and 11 being especially good candidates. For example, Ni and Pt catalysts provide strong catalysis for the present approach.

The MF and/or MFA reduction reaction proceeds in the presence of hydrogen. The hydrogen pressure may be at or above atmospheric pressure, and may be, for example, at 100 psig, 200 psig, 300 psig, 400 psig, 500 psig, 600 psig, 700 psig, 800 psig, 900 psig, or 1,000 psig, or any increment between those values. In preferred embodiments the hydrogen pressure is at least 500 psig, and may be even higher depending on the catalyst material, the reactor, and equipment used in the particular embodiment. Hydrogen is present at a molar ratio greater than 1.0, relative to the furan (MF or MFA), but it should be appreciated that the optimum ratio for a given embodiment may be determined through the use of routine skill in the art.

In some embodiments using either Pd/C or Pd/Al₂O₃ as the catalyst, a tertiary amine may be incorporated to extend the catalyst life. However, the tertiary amine is not effective for extending the life of the bulk Cu catalyst. In some embodiments, the amine is no more than 5.0 wt. % of the solution (reactant in organic solvent), and may be less than 4.5 wt. % of the solution, or less than 4.0 wt. % of the solution, or less than 3.0 wt. % of the solution, or less than 2.0 wt. % of the solution, or less than 1.0 wt. % of the solution, or any increment of 0.1 wt % thereof.

Tributylamine (TBA) suppresses ring opening and hydrogenation activity. This is particularly noticeable with noble metals. Hypothesized that the TBA passivated the acid sited needed to do ring opening chemistry.

The following paragraphs describe examples of the present approach for producing DMF as described above. It should be appreciated that deviations may be made to the specific examples based on this disclosure and the level of ordinary skill in the art without departing from the present approach.

The demonstrative runs took place via a batch process involving a stirred tank reactor and a packed bed reactor. The reaction in the stirred tank reactor proceeded in the liquid phase and the reaction in the packed bed reactor proceeded in both the vapor phase and the liquid phase. It should be appreciated that the reactions may proceed continuously. Additionally, the reactions of the present approach may be performed in a batch reactor, a tubular reactor, a continuous stirred tank reactor (CSTR), or combinations thereof.

In one series of demonstrative runs, DMF was reacted with ethylene in the presence of trifluoromethanesulfonic acid (also known as triflic acid) and different organic solvents to produce pX. The organic solvents evaluated included, e.g., toluene, dodecane, tetradodecane (Parafol-14, Sasol Chemicals), Isopar™-L, and Isopar™-M (ExxonMobil Chemical Co.). Each solvent was evaluated at reducing DMF concentration in the feed to a reactor. All solvents evaluated provided similar DMF conversion and pX selectivity. This demonstrates that nearly any organic solvent is effective for the reaction, provided that the solvent is inert to the Diels-Alder chemistry. It should be appreciated that in preferred embodiments, the organic solvent is heavier than pX, to allow for recovery of a high-purity pX product.

Advantageously, the present approach allows for selecting reaction conditions that promote extended catalyst life and achieve target conversions for reducing MF and MFA. It should be appreciated that in some embodiments, the initial reactant is MF, whereas in some embodiments the initial reactant is MFA, and in other embodiments the initial reactant is a combination of MF and MFA. Hydrogenation of MF can proceed at low temperatures, such as 50-300° C. and preferably at a temperature of about 150-190° C., including any 10° C. increment within that range and is not the rate determining step in the present approach. Instead, hydrogenolysis of MF A is the rate determining step and proceeds at higher temperatures. Selectivity of DMF improves as the reaction temperature is increased past 170° C., and preferably the reaction proceeds at about 180-240° C., and more preferably at about 190-230° C. The reduction of MF and MFA may proceed at a pressure of above 0 to 1,000 psig. Hydrogen may be maintained at a pressure of above 0 to 1,000 psig, and preferably above about 500 psig to 1,000 psig in the reactor. Additionally, the reaction can proceed in the liquid phase and/or the vapor phase. Hydrogen solubility and reactivity can be improved at higher pressures in the liquid phase and drives the reaction forward. In the disclosed examples, baseline reactions proceeded at 500 psig, but it should be appreciated that they can proceed at 100 psig, 200 psig, 300 psig, 400 psig, 500 psig, 600 psig, 700 psig, 800 psig, 900 psig, or 1,000 psig, for example. In preferred embodiments, the pressure is at least 500 psig. Preferably, the reaction proceeds at a temperature in the range of about 190 to 240° C., but it should be appreciated that the optimum temperature will depend on the catalyst used in particular embodiment. In some embodiments, an amine is added to the MF and/or MFA feed to increase DMF selectivity and reduce ring hydrogenation and ring opening chemistry. The amine also beneficially extends the catalyst life and service factor for continuous reactor units. Some embodiments may feature an additional separation step following the reduction of MF and/or MFA, to produce a purified DMF product.

The conversion of DMF to pX proceeds at an operating temperature of about 150° C. to 300° C., or any 10° C. increment in that range. The operating pressure for 2,5-dimethylfuran to p-xylene conversion is from about 500 to 2000 psig, but the reaction may proceed at any pressure within that range, e.g., 700 psig, 1,000 psig, 1,200 psig, 1,400 psig, 1,600 psig, 1,800 psig, etc. As referenced above, this reaction also proceeds with a catalyst. Preferably the catalyst is a strong acid.

The following paragraphs describe observations made with prototype embodiments of the present approach using a fixed bed apparatus, and provide guidance for practicing the present approach based on the results discussed below.

DMF selectivity strongly correlates with reaction temperature. Although selectivity for DMF is dependent on conversion, a stronger impact is observed with temperature. Embodiments of the present approach preferably react at 170° C. to 240° C. Lower temperatures favor MFA over DMF.

The incorporation of amine had a more profound effect for Pd catalysts. The amine demonstrated longer time on stream without need for regeneration. Both experiments shown below had equivalent productivity quantified as turnover number, TON, and is defined as grams DMF/grams Pd. A tie point under similar conditions and conversions is also shown and exhibits a lower deactivation rate and higher selectivity for DMF. Without the amine a higher level of ring-based chemistry is observed. These reactions include ring hydrogenation and ring opening reactions. The unwanted byproducts are 2,5-hexanedione and its hydrogenated byproducts and 2,5-dimethyltetrahydrofuran.

There is a direct correlation between hydrogen pressure and conversion. This effect is more prominent in the liquid phase. Hydrogen solubility can be a limiting factor in the liquid phase for some embodiments. Higher pressures may be used to drive conversion forward and reduce reactive species that can lead to foulants. The vapor phase showed a very high increase in conversion (full conversion). The transition happened mid run and increased conversion from 30% to full. It is likely that hydrogen and MF had higher accessibility to the catalyst and increased the rate of conversion. However, a steep deactivation was observed, possibly from heavy species forming in the vapor phase and depositing on the catalyst.

The following paragraphs describe observations made with prototype embodiments of the present approach using a batch stirred tank reactor, and provide guidance for practicing the present approach based on the results discussed below.

With respect to catalyst selection, reaction temperature, and reaction time, as shown in reaction scheme [II] above, the production of DMF from MF can be treated as two sequential reactions in which MFA is the primary product and DMF the secondary product.
1. Aldehyde hydrogenation of MF to MFA
2. Alcohol hydrogenolysis of MFA to DMF

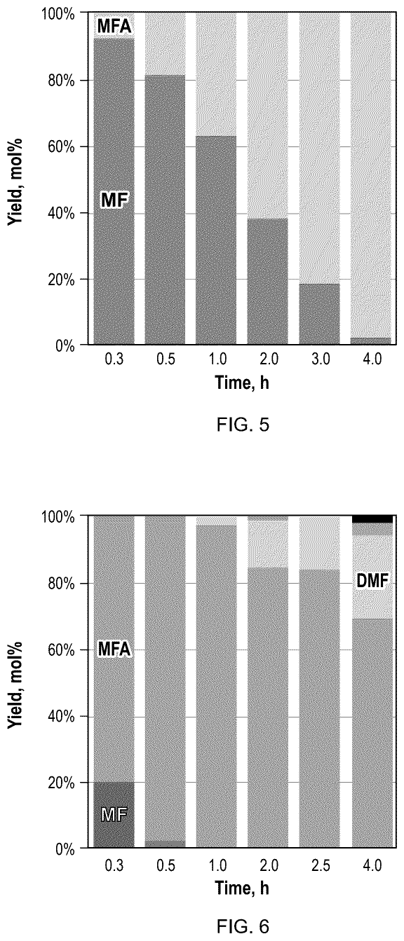

The second reaction is expected to have a higher activation barrier than the first and, therefore, be slower. This relative difference in reaction rates can be exploited to preferentially produce MFA by using less active catalyst, lowering the reaction time, or lowering the reaction temperature. Silica-supported bulk Cu catalysts, such as Johnson-Matthey 60/08P are less active and preferentially promote aldehyde hydrogenation over alcohol hydrogenolysis. FIG. 5 shows the molar yield of MF hydrogenation over JM 60/08P at 170° C. as a function of time. After 4 h, 97% of the starting MF is consumed with 100% selectivity for MFA.

In alternative embodiments, similar yields of MFA can be reached with the same catalyst at higher temperatures and shorter reaction times. FIG. 6 shows MF, MFA, and DMF yield over time at 230° C. After 30 minutes, the conversion of MF is 98% and the selectivity to MFA is 100%. At 230° C. and 1 hour, the conversion of MF is 100% and the selectivity to MFA is 97%. It should be appreciated that catalyst selection, reaction time, and reaction temperatures can be selected to preferential produce MFA with high yields.

The following paragraphs describe prototype embodiments of the present approach using a batch stirred tank reactor for producing DMF and provide guidance for practicing the present approach based on the results discussed below.

In some embodiments, Pd is the preferred catalyst for MF-to-DMF. Pd had shown higher activity than Cu-based catalysts in the continuous process and its selectivity to DMF was high when passivated with TBA. However, in the batch reactor Pd/Al₂O₃ and Pd/C had poor activity in the hydrogenation of dilute MF and low selectivity to DMF. On the other hand, CuO/ZnO bulk powder catalyst, showed complete conversion of dilute MF and high selectivity to DMF. In the tests described above, the MF-to-metal molar ratio was over two orders of magnitude higher for Cu relative to Pd. The two reactions used 2 g of ˜66 wt % CuO/ZnO catalyst and 0.15 g of 5 wt % Pd/C for 14 g of MF. However, the cost of Pd is over 3,000 times the cost of Cu on a molar basis. It should therefore be appreciated that Cu-based catalysts should be considered for batch reactor hydrogenation of MF to DMF.

The effect of initial MF concentration on DMF production was tested for 5 wt % MF and 40 wt % MF. The hydrogenation was carried out for 4 h at 170° C. and 500 psi using BASF Cu-0313P as the catalyst. The MF-to-Cu ratio was 10:1 for the 5 wt % MF reaction and 100:1 for the 40 wt % MF reaction. The conversion of MF was 100% for the high and low starting concentrations. However, the lower starting MF concentration favored the production of DMF relative to the higher starting MF concentration. Indeed, the selectivity to ring-hydrogenation products, such as DMTHF, was also for the lower starting MF concentration. This is consistent with a higher MF-to-Cu ratio promoting deeper hydrogenation products. Finally, no solids or other polymerization products were observed for the higher starting MF concentration. In fact, the C6 molar balance improved. Higher initial MF concentrations can be used in embodiments of the present approach, but a higher reaction temperature and longer reaction time may be needed to produce DMF.

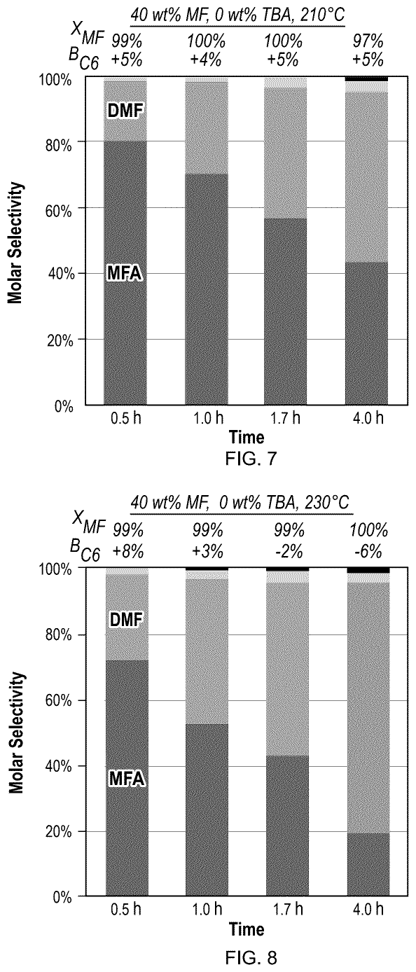

The slower MFA to DMF reaction is reflected in conversion of MF and selectivity to MFA and DMF over time. FIGS. 7 and 8 show molar selectivity of MF hydrogenation over BASF-0313P at 210° C. and 230° C., respectively, over 4 h. After 30 min at 210° C., all MF is consumed and the selectivity to MFA is 80 mol %. Over the next 3.5 h, the selectivity to DMF increases from 20 mol % to 55 mol % as the MFA is slowly consumed. The same trends appear at 230° C., but the production of DMF is more favored. Thus, longer residence times favor higher selectivity to DMF in embodiments of the present approach.

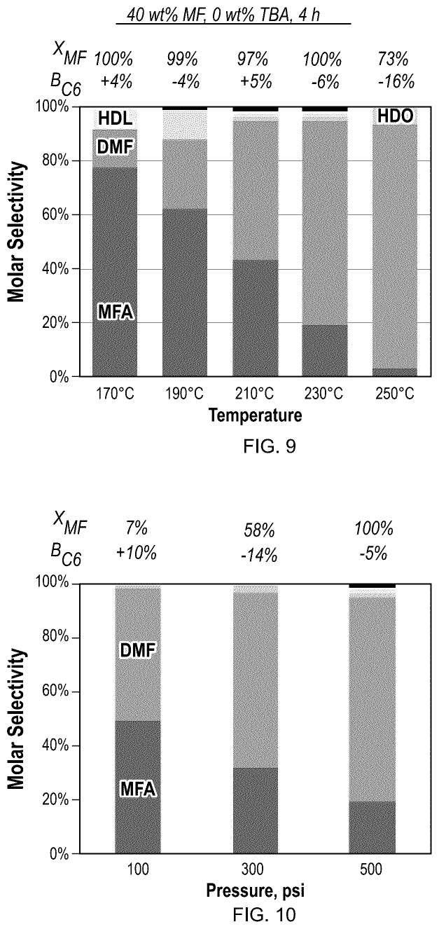

FIG. 9 shows molar selectivity over various reaction temperatures. For nearly all temperatures tested, the conversion of 40 wt % MF was complete in 4 h. At 210° C. and 250° C., MF conversion was complete by 30 minutes of reaction time. However, the final selectivity to DMF at 4 h increased with temperature from 13 mol % at 170° C. to 89 mol % at 250° C. As discussed above, the MF readily hydrogenates to MFA under the conditions in this study, but the hydrogenolysis of MFA to DMF is slower. The higher temperatures increase the selectivity to DMF by increasing the rate of the second reaction. Thus, it should be appreciated that higher temperatures favor higher selectivity to DMF in embodiments of the present approach.

FIG. 10 shows molar selectivity as a function of hydrogen pressure in the production of DMF according to the present approach. As seen in FIG. 10, increasing H₂ pressure increases the conversion of MF and the selectivity to DMF after 4 h at 230° C. over a bulk CuO/ZnO catalyst. Higher hydrogen pressures therefore favors higher selectivity to DMF and higher reaction rates in the present approach.

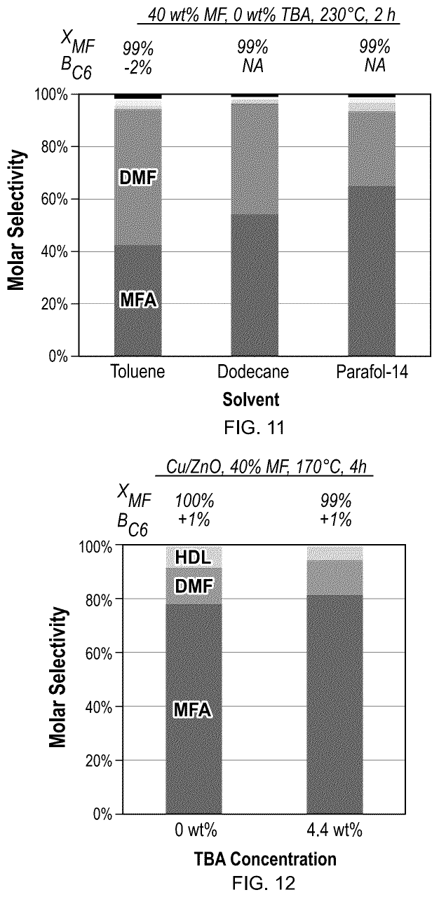

In some embodiments for producing pX, the product from the MF-to-DMF reactor is used without further purification in the DMF-to-pX reactor. In such embodiments, the solvent should be heavier than pX for an easier distillation. Toluene does not meet that requirement, so dodecane and Parafol-14 were tested. FIG. 11 shows molar selectivity for different solvents. The selectivity to DMF after two hours was lower with dodecane and Parafol-14 solvents, likely due to a decrease in the hydrogenation rates. With toluene, all MF is converted by 30 minutes of reaction time. In dodecane and Parafol-14, MF conversion is not complete until 1 h. This rate decrease also applies to the conversion of MFA to DMF. The lower rates in dodecane and Parafol-14 mean that the ultimate selectivity to DMF is lower in both solvents. The lower reaction rates in paraffin solvents might be due to mass transport limitations, as two organic phases are present in the reactor. As such, a heavy aromatic solvents (1-methyl naphthalene) are being evaluated in further demonstrations of the present approach. The data show that aromatic solvents can be considered to prevent phase separations that lower reaction rates.

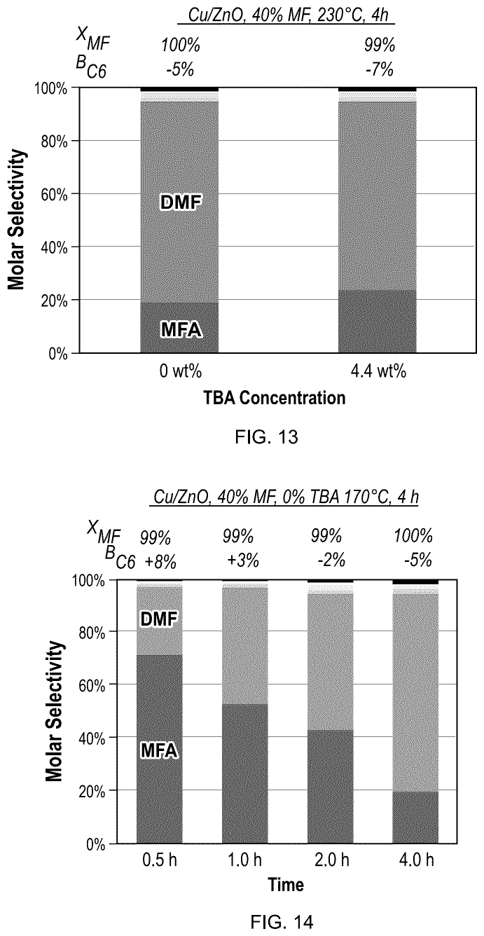

In prototype embodiments, TBA was included in the reaction mixture as it had proved useful in the continuous MF-to-DMF process. FIGS. 12 and 13 show molar selectivity in DMF production for different concentrations of TBA, at 170° C. and 230° C., respectively. In these embodiments, Pd/Al₂O₃ was the preferred catalyst and the inclusion of TBA lowered the selectivity to ring-hydrogenation products by, it was argued, passivating the catalyst surface. The inclusion of TBA in the MF-to-DMF process introduces the need for a separation or neutralization step prior to the DMF-to-pX process. However, MF has a 9:1 MF-to-TBA mass ratio and that amine would therefore be easier to separate from the product of MF-to-DMF rather than the feed.

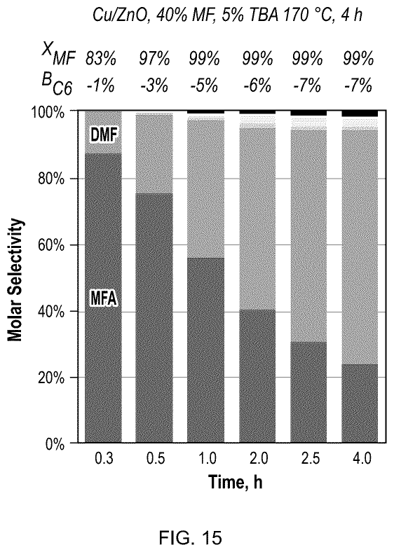

As FIGS. 12 and 13 show, the inclusion of TBA in the batch hydrogenation of MF results in a minor decrease in the selectivity to DMF after 4 h of reaction. This is the case at 170° C. and 230° C. At the lower temperature, the main product is MFA and the selectivity towards DMF is ˜10 mol % with and without TBA. At the higher temperature, the main product is DMF and the selectivity to MFA is ˜20 mol % with and without TBA. FIGS. 14 and 15 show molar selectivity as a function of time, with and without TBA as the amine, respectively. Furthermore, as shown below, the reaction rates over 4 h are also minimally affected by the inclusion of TBA. As can be seen, TBA does not alter the activity or selectivity of BASF 0313-P between 170° C. and 230° C. in embodiments of the present approach. The decision to include TBA in the feed should be made based on separation and operation constraints.

The terminology used herein is for the purpose of describing particular embodiments only and is not intended to be limiting of the approach. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, or components, but do not preclude the presence or addition of one or more other features, integers, steps, operations, elements, components, and/or groups thereof.

The present approach may be embodied in other specific forms without departing from the spirit or essential characteristics thereof. The present embodiments are therefore to be considered in all respects as illustrative and not restrictive, the scope of the invention being indicated by the claims of the application rather than by the foregoing description, and all changes which come within the meaning and range of equivalency of the claims are therefore intended to be embraced therein.

TABLE 3
Data for converting CMF to MF according to embodiments
of the present approach and contemporary processes.

| | | | | | | Amine:CMF | CMF |
|---|---|---|---|---|---|---|---|
| | Reactor | | | | | Ratio | Concentration |
| Run | Scale | | CMF Source | Solvent | Amine | (mol/mol) | (wt %) |
| 1 | 50 | L | Cornstarch, Southern Pine | Toluene | TBA | 1.3 | 9% |
| 2 | 50 | L | Southern Pine | Toluene | TBA | 1.3 | 7% |
| 3 | 0.3 | L | Crystalline CMF | Toluene | TBA | 1.1 | 10% |
| 4 | 0.3 | L | Sucrose | Toluene | TBA | 1.1 | 5% |
| 5 | 1 | L | Cornstarch, Hardwood | DMFD | Hunio's Base | 1.1 | 10% |
| 6 | 1 | L | Cornstarch, Hardwood | DMFD | Hunio's Base | 1.1 | 10% |
| 7 | 1 | L | Crystalline CMF | Dowtherm A | Alamine 336 | 1 | 5% |
| 8 | 1 | L | Crystalline CMF | Dowtherm A | Alamine 304 | 1 | 5% |
| 9 | 1 | L | Crystalline CMF + TBA [symbol] | DMFD | TBA | 1 | 4% |
| 10 | 1 | L | Crystalline CMF + TBA [symbol] | Water | TBA | 1 | 2.50% |
| 11 | 1 | L | Crystalline CMF | Aromatic | TEHA | 1.3 | 20% |
| | | | | 200 ND | | | |
| 12 | 1 | L | Crystalline CMF | Aromatic | TEHA | 1.3 | 20% |
| | | | | 200 ND | | | |
| 13 | 1 | L | Crystalline CMF | Aromatic | TEHA | 1.3 | 20% |
| | | | | 200 ND | | | |
| 14 | 1 | L | Cornstarch, Hardwood | Toluene | TIBA | 1.1 | 13% |
| 15 | 1 | L | Cornstarch, Hardwood | Toluene | TAA | 1.1 | 13% |
| 16 | 1 | L | Cornstarch, Hardwood | Dowtherm A | DEAN | 3 | 7% |
| 17 | 15 | mL | Fructose? | Toluene | pyridine | 1 | 10% |
| 18 | 15 | mL | Fructose? | Toluene | triethylamine | 1.5 | 10% |
| 19 | 15 | mL | Fructose? | Chloroform | triethylamine | 1 | 6% |
| 20 | 100 | mL | Crystalline CMF | Toluene | triethylamine | 1.5 | 10% |
| 21 | 100 | mL | Sucrose | Toluene | triethylamine | 1.5 | 10% |
| 22 | 100 | mL | Sucrose | Toluene | triethylamine | 1.5 | 10% |
| 23 | 0.3 | L | Sucrose | Toluene | triethylamine | 1.5 | 10% |
| 24 | 0.3 | L | Sucrose | Toluene | triethylamine | 1.5 | 10% |

| | CMF TON | H [symbol] | | Reaction | CMF | MF | MF TON |
|---|---|---|---|---|---|---|---|
| | (mol CMF/ | Pressure | Temp | Time | Conversion | Yield | (mol MF/ |
| Run | mol Pd) | (psia) | (° C.) | (h) | (mol %) | (mol %) | mol Pd) |
| 1 | 704 | 215 | 99 | 3 | 99% | 91% | 640 |
| 2 | 720 | 215 | 107 | 3 | 100% | 93% | 670 |
| 3 | 147,000 | 315 | 120 | 4.25 | 80% | 69% | 102,000 |
| 4 | 7,400 | 315 | 120 | 0.5 | 100% | 99% | 7,300 |
| 5 | 7,400 | 315 | 120 | 1.5 | 97% | 97% | 7,100 |
| 6 | 50,000 | 315 | 120 | 15 | 100% | 85% | 42,400 |
| 7 | 9,300 | 315 | 120 | 20 | 100% | 87% | 8,100 |
| 8 | 9,300 | 315 | 120 | 27 | 100% | 81% | 7,500 |
| 9 | 18,400 | 315 | 120 | 14 | 100% | 75% | 13,800 |
| 10 | 7,400 | 315 | 120 | 14 | 100% | 85% | 6,300 |
| 11 | 18,300 | 515 | 120 | 3.5 | 100% | 97% | 17,800 |
| 12 | 18,400 | 115 | 120 | 6 | 99% | 91% | 16,700 |
| 13 | 18,500 | 315 | 80 | 16 | 100% | 89% | 16,500 |
| 14 | 7,300 | 315 | 120 | 14 | 80% | 69% | 5,100 |
| 15 | 7,400 | 315 | 120 | 18 | 100% | 80% | 5,900 |
| 16 | 18,400 | 315 | 90 | 17 | 100% | 92% | 17,000 |
| 17 | 20 | 15 | 40 | 3.5 | 100% | 94% | 19 |
| 18 | 22 | 15 | 35 | 2.5 | 100% | 99% [symbol] | 22 |
| 19 | 20 | 15 | 35 | 3 | 100% | 94% | 19 |
| 20 | 22 | 15 | 35 | 2.5 | 100% | 61% [symbol] | 14 |
| 21 | 200 | 15 | 35 | 22 | 100% | 91% | 182 |
| 22 | 1000 | 15 | 35 | 22 | 32% | 8% | 80 |
| 23 | 1000 | 315 | 120 | 1.5 | 99% | 75% | 750 |
| 24 | 5000 | 315 | 120 | 17 | 100% | 47% | 2,350 |

[symbol]

indicates data missing or illegible when filed

What is claimed is:

1. A process for converting 5-chloromethylfurfural, the process comprising:
reacting a 5-chloromethylfurfural (CMF) stock in an organic solvent and an amine base additive in the presence of hydrogen over a Pd catalyst to produce an effluent having at least one of 5-methyl furfural (MF) and 5-methyl furfuryl alcohol (MFA), and at least one amine-HCl salt.

2. The process of claim 1, wherein the organic solvent is toluene and the amine base additive is tri-n-butyl amine.

3. The process of claim 1, wherein the Pd catalyst is initially present at a molar ratio of 5,000 to 150,000 CMF to Pd catalyst.

4. The process of claim 1, wherein the reaction proceeds at at least one of a temperature of 50° C. to 150° C. and a hydrogen pressure of 115-515 psia.

5. The process of claim 1, further comprising neutralizing the at least one amine-HCl salt to produce a composition having at least one of MF and MFA, the organic solvent, and a neutralized amine.

6. The process of claim 5, wherein the at least one of MF and MFA is MF, and further comprising decanting the MF and neutralized amine composition to produce an MF-rich phase and an amine-rich phase.

7. The process of claim 6, further comprising distilling the amine-rich phase to produce a purified amine and an amine-MF distillate and recycling the amine-MF distillate to a decanting process.

8. The process of claim 6, further comprising distilling the MF-rich phase to produce a purified MF and an amine-MF distillate and recycling the amine-MF distillate to a decanting process.

9. The process of claim 5, wherein the at least one amine-HCl salt is neutralized through at least one of: a caustic agent, an inorganic base, sodium hydroxide (NaOH), sodium bicarbonate (NaHCO₃), magnesium hydroxide (Mg(OH)₂), calcium oxide (CaO), and calcium hydroxide (Ca(OH)₂).

10. The process of claim 6, wherein the 5-chloromethylfurfural stock further comprises organic soluble materials (OSMs).

11. The process of claim 10, wherein the OSMs remain in a solution with MF, and the process further comprising separating the MF and the OSMs using a heavy solvent to prevent or minimize OSM solidification.

12. The process of claim 1, wherein the reaction proceeds in at least one batch reactor or in at least one continuous reactor.

13. The process of claim 1, wherein the amine base additive is an amine having the formula R₃N, wherein each R may be the same or different and is a straight chain alkyl or a branched chain alkyl, provided that the average number of carbon atoms in each R is from 1 carbon atoms to 12 carbon atoms.

14. The process of claim 1, wherein the amine base additive is at least one amine selected from the group consisting of: tri-n-butyl amine (TBA), tri-isobutyl amine (TiBA), tris-2-ethylhexyl amine (TEHA), trihexylamine (THEX), dimethyldocecylamine (DIMLA), dibutylaniline (DBAN), trimethylamine (TMA), N, N-diisopropylethylamine (DIPEA), tri-n-amylamine, tri-octylamine, and triethylamine (TEA).

15. The process of claim 2, wherein the 5-chloromethylfurfural stock comprises CMF and at least one organic solvent selected from the group consisting of a polar organic solvent, methanol, acetone, dimethylformamide, MF, tetrahydrofuran, furfural, dimethylfuran, a non-polar organic solvent, toluene, benzene, alkylbenzene, and xylene.

16. The process of claim 1, wherein the 5-chloromethylfurfural stock comprises a CMF concentration of less than 60 wt %, or less than 50 wt %, or less than 40 wt %, or less than 30 wt %.

17. The process of claim 1, further comprising reducing the at least one of MF and MFA in a solvent in the presence of hydrogen and a metal catalyst to produce a 2-5-dimethylfuran (DMF) effluent.

18. The process of claim 17, further comprising reacting the DMF effluent with ethylene in the presence of an acid catalyst to produce para-xylene.

19. The process of claim 5, wherein the at least one of MF and MFA is MFA, and further comprising separating the MFA and neutralized amine by at least one of decantation, liquid-liquid extraction, and distillation, to produce an MFA-rich phase and an amine-rich phase.

20. The process of claim 1, further comprising neutralizing the effluent with an aqueous base to produce a neutralized mixture of the at least one of MF and MFA, the organic solvent, a neutralized amine, and at least one aqueous chloride salt.

21. The process of claim 20, further comprising separating the neutralized mixture by at least one of decantation, liquid-liquid extraction, and distillation, to produce an amine-rich phase and at least one of an MF-rich phase and MFA-rich phase.

22. The process of claim 20, further comprising reacting the effluent with aqueous HCl to produce an acid-washed mixture of an organic phase having the at least one of MF and MFA and the organic solvent, and an aqueous phase having an amine-HCl salt.

23. The process of claim 22, further comprising decanting the acid-washed mixture to produce an organic phase having the at least one of MF and MFA and the organic solvent, and an aqueous phase having amine base additive; and
neutralizing the aqueous phase with a base to produce a recovered amine.

24. The process of claim 23, further comprising separating the organic phase to produce an organic solvent distillate product and a bottoms product with the at least one of MF and MFA.

25. The process of claim 24, further comprising separating the bottoms product to produce a high-purity product.

26. The process of claim 1, further comprising neutralizing the effluent with a solid base to produce a neutralized mixture of the at least one of MF and MFA, the organic solvent, and a neutralized amine.

27. A process for producing 5-methyl furfural (MF), the process comprising:
reacting a 5-chloromethylfurfural (CMF) stock having CMF, organic soluble materials (OSMS), and an organic solvent, with an amine base additive in the presence of hydrogen over Pd catalyst initially present at a molar ratio of 5,000 to 150,000 CMF to Pd catalyst, to produce an effluent having MF, OSMs, the organic solvent, and at least one amine-HCl salt, wherein the reaction proceeds at at least one of a temperature of 50° C. to 150° C. and a hydrogen pressure of 115-515 psi;
thermally cracking the effluent to produce a recovered HCl product, an organic solvent distillate product, and an MF-rich bottoms product having MF, a recovered amine, and OSMs;
distilling the MF-rich bottoms product to produce an MF distillate product and an amine-rich bottoms product containing the amine base additive and the OSMs;
distilling the amine-rich bottoms product to produce an amine base additive distillate produce and an OSMs bottoms product.
